(19) United States
(12) Patent Application Publication
(10) Pub. No.: US 2008/0287729 A1
(43) Pub. Date: Nov. 20, 2008

(54) INTERSTITIAL MARKER AND METHOD FOR CREATION THEREOF

(76) Inventors: Anthony Biscotti, Acton, MA (US); Bruce Horowitz, Palm Beach Gardens, FL (US)

Correspondence Address: LAW OFFICE OF BRETT N. DORNY, 386 WEST MAIN STREET, SUITE 12A, NORTHBOROUGH, MA, 01532, US

(21) Appl. No.: 11/749,317

(22) Filed: May 16, 2007

(51) Int. Cl. A61M 36/00

(52) U.S. Cl. 600/8

(57) ABSTRACT

An interstitial marker for localization of organs, biopsy sites, tumors or tumor beds, is comprised of a fluid marker material which is visible under at least one imaging modality. The fluid marker material is injected at the desired location to create a marker having a desired size and configuration. The configuration includes combinations of straight and curved lines, spheres and blank spaces. The fluid marker material includes a high density metal such as bismuth, gold, or iridium suspended in a liquid, in colloidal form or made into a solution.

### BACKGROUND OF THE INVENTION

1. Field of the Invention

The present invention relates to interstitial markers for localization of organs, tumors or tumor beds. More particularly, it relates to an interstitial marker of a high density metal in a solution formed through injection.

2. Discussion of Related Art

Interstitial markers have long been known to the medical world. They are regularly used to prepare patients undergoing radiation treatment. Such treatments are typically split into daily doses or fractions. The interstitial markers help the radiation clinician target the therapy to the same area each time treatment is performed on a given patient's tumor. Interstitial markers are also used in tracking a biopsy site and tracking organ or tumor volume changes, either increases or decreases, over time.

Historically interstitial markers have been produced of metal “seeds” pre-shaped in the form of a grain of rice or a pre-formed metal coil. Known markers have a standard shapes and sizes. The marker cannot be adjusted based upon the specific needs for the individual. Also, the shape and size of the marker are selected prior to insertion. They cannot be adjusted during positioning. The pre-formed marker is implanted at the desired location through injection. To implant a large diameter marker into a given tissue bed, a large bore needle, such as 18 gauge, must be used. Such a large needle requires anesthesia be use on the patient which complicates the procedure.

### SUMMARY OF THE INVENTION

The present invention substantially overcomes the deficiencies of known interstitial markers through the use of a liquid marker material. According to various aspects of the invention, bismuth, gold and/or iridium particles are used in the formation of the liquid marker material. According to other aspects of the invention, the metal particles are suspended in a fluid of dextrose, saline or silicon. According to other aspects of the invention, particles of biocompatible plastics or glass micro beads are used in the formation of the liquid marker material. According to another aspect of the invention, the fluid includes Vitamin E oil.

According to another aspect of the invention, the liquid marker material is injected into the body of the patient at the desired marker location. According to another aspect of the invention, the needle used for injecting the liquid marker is moved during the injection process to create a specific marker shape. According to another aspect of the invention, the needle includes a drive mechanism for precise deposition of the liquid marker material.

### BRIEF DESCRIPTION OF THE DRAWINGS

FIG. 1 is an illustration of a needle for delivery of the interstitial marker according to an embodiment of the present invention.

FIG. 2 is an illustration of a needle for delivery of the interstitial marker according to another embodiment of the present invention.

FIGS. 3A-3G illustrate representative interstitial markers created according to an embodiment of the present invention.

### DETAILED DESCRIPTION

An interstitial marker according to an embodiment of the present invention is formed by metal particles suspended in a fluid. Preferably, the metal particles are of bismuth, gold or iridium. These metals are easily visible when viewed via MRI (Magnetic resonance imaging), PET (positron emission tomography), CT (computerized tomography), Digital X-ray radiography, mammography, and ultrasound. Bismuth subnitrate and bismuth subcarbonate are used extensively in medicine and have a long history of safety. Of course, other metals or materials which would be visible using any desired technology could be used. When the interstitial marker is to be viewed with MRI, materials other than metals are preferable. MRI technology does not image gold well. It does, however, provide images of plastics and glass. Therefore, particles of biocompatible plastics, such as high molecular weight polyethylene, or other materials visible using MRI technology can be used for the interstitial marker. Similarly, micro beads of glass could also be used. Furthermore, multiple types of particles can be used with the interstitial marker. The types of particles can be selected for their imaging characteristics using different technologies. With multiple types of particles, the interstitial marker may be easily viewed with different imaging technologies.

The metal or other particles are approximately 5 microns to 25 microns in diameter. In one embodiment of the invention, the particles have a diameter of 10 microns. Of course, other dimensions could be used. Smaller particles tend to be taken up by the tissue rather than remain in place. Larger particles can cause clots in blood vessels. The particles may have uniform diameters, but need not be uniform. Furthermore, the particles do not need to have uniform shapes.

In the present invention, the metal particles are suspended in a fluid. Preferably, the fluid is saline, dextrose solution, dimethly-silicone or other silicon based solution. Of course, other fluids may be used. The fluid may provide additional benefits for viewing the interstitial marker with different imaging technologies. For example, the fluid may include vitamin E oil which is easily visible with MRI technology. In one embodiment, a 5% dextrose solution is used for the fluid. According to an embodiment of the invention, concentrations of approximately 1 to 2 grams of gold particles to a milliliter of fluid are used. Similar concentrations of other metals may also be used, as well as different concentrations.

An interstitial marker is created by injecting the suspension into the patient at the desired location. Since the metal particles are small, a small gauge needle (29 gauge) can be used, regardless of the desired marker size. The size of the marker is determined by the amount of suspension injected at a location. Furthermore, the needle can be moved during the injection process to create different markers of various shapes and sizes.

An added advantage of a liquid marker system is that the same micro needle can implant a marker in a fine or coarse line form, larger or smaller spheres attached to a line or any combination required by the user. The small gauge needle is easier to “steer” in tissue allowing for larger arches in the marker material creating a marker that has a better ability to create the three dimensional image when viewed to more accurately plan for patent treatment.

The ability to use a small gauge needle is also beneficial to treatment of the patient. Anesthesia is not required with the small gauge needle. This makes the procedure faster, less costly, and safer for the patient. The patient has less bleeding. Also, the chances of allergic or adverse reactions are reduced. The use of a small gauge needle also allows interstitial markers to be placed at locations not previously possible. For example, the interstitial marker of the present invention can be injected into visceral and vascular areas which could be damaged by larger needles, such as the stomach, pancreas, lung and brain.

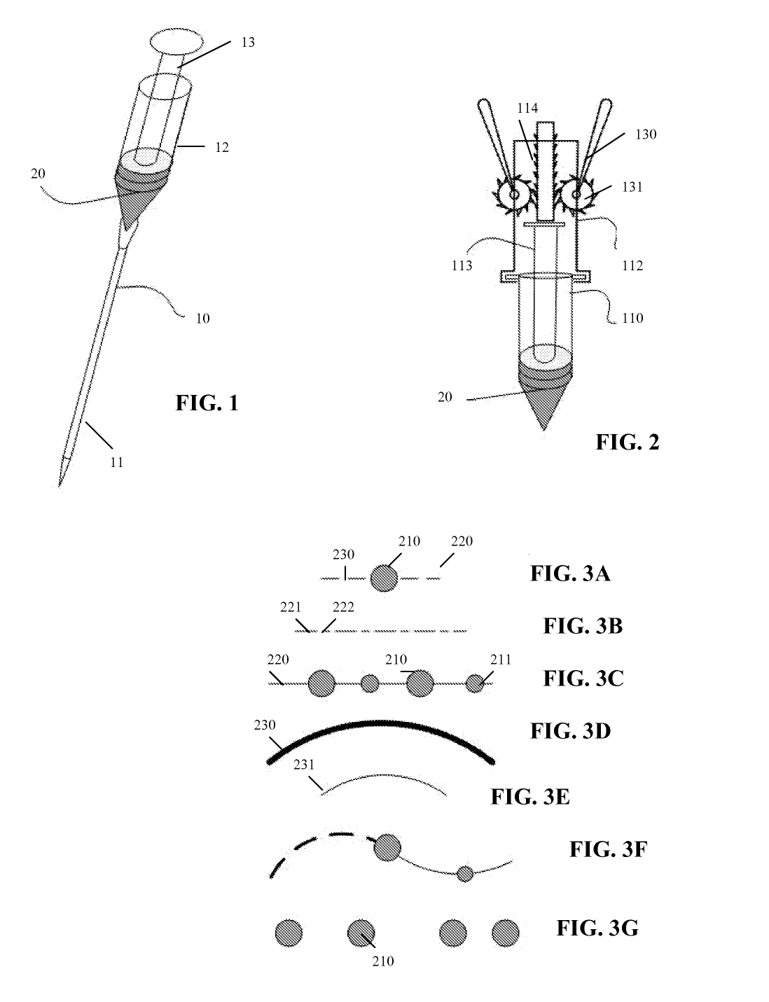

FIG. 1 is a perspective view of a standard needle 10 which can be used to create an interstitial marker according to an embodiment of the present invention. The needle 10 includes a small gauge needle 11 and a syringe 12. According to embodiments of the invention, 21 or 29 gauge needles are used. Of course, other size needles which can accommodate the metal particles could also be used. The metal suspension 20 is placed in the syringe. The needle 11 is inserted into the patient's body so that the tip is at the desired location for the marker. Imaging techniques can be used to accurately position the tip of the needle. The plunger 13 is pressed to inject the metal suspension 20 into the patient. The operator can move the tip of the needle to create an interstitial marker of a desired size and shape.

FIG. 2 illustrates another needle 110 for injecting the metal suspension 20 to create an interstitial marker according to another embodiment of the present invention. As in FIG. 1, the needle 110 includes a small gauge needle (not shown) and a syringe 112. The plunger 113 includes a plurality of teeth 114 positioned on one or more sides. A set of drivers 130 include toothed wheels 131 to engage the teeth 114 of the plunger 113. A motor (not shown) rotates the wheels 131 to depress the plunger 113. With an appropriate motor, this embodiment allows accurate control of the amount of metal suspension 20 which is injected.

FIGS. 3A-3G illustrate possible marker shapes. A large variety of shapes can be created. This allows optimal markers to be created for specific locations or needs. With the present invention, any shape or size marker can be created during the injection process. FIG. 3A illustrates a larger sphere 210 with narrow lines 220 on either side. A sphere 210 is created by injecting more of the metal suspension while moving the needle slowly. A narrow line 220 is created my injecting less of the suspension while moving the needle more quickly. Empty spaces 230 can also be create by moving the needle without injecting any of the metal suspension.

FIG. 3B illustrates forming multiple narrow lines 221, 222. The lines may be long 221 or short 222, or various combinations thereof. FIG. 3C illustrates spheres of various sizes 210, 211 connected with narrow lines 220. FIGS. 3D and 3E illustrate curved marker shapes. The curved lines may be thick 230 or thin 231 depending upon the rate of injection of the metal suspension relative to the speed of needle movement. FIG. 3F illustrates a complex marker shape which can be created in accordance with the present invention. Alternatively, a simple set of spheres may be created, as illustrated in FIG. 3G.

Having disclosed at least one embodiment of the present invention, various adaptations, modifications, additions, and improvements will be readily apparent to those of ordinary skill in the art. Such adaptations, modifications, additions and improvements are considered part of the invention which is only limited by the several claims attached hereto.

1. An interstitial marker for injection within a mammalian body comprising:
a plurality of particles visible under at least one imaging modality; and
a fluid in which the plurality of particles are suspended.

2. The interstitial marker according to claim 1 wherein the plurality of particles and fluid are injected within the mammalian body to create a specific marker size and configuration.

3. The interstitial marker according to claim 2 wherein the marker size and configuration are determined by movement of a needle used for injecting the marker.

4. The interstitial marker according to claim 2 wherein the marker configuration includes at least one of sphere.

5. The interstitial marker according to claim 2 wherein the marker configuration includes at least one line.

6. The interstitial marker according to claim 3 wherein the marker configuration includes at least two elements separated by a space.

7. The interstitial marker according to claim 2 wherein the size of the marker is greater than an inside diameter of a needle used to inject the marker.

8. The interstitial marker according to claim 1 wherein the plurality of particles include at least one of gold, bismuth, and iridium.

9. The interstitial marker according to claim 1 wherein the marker may be injected into the mammalian body using a 30 gauge or larger needle.

10. The interstitial marker according to claim 1 wherein the plurality of particles include a radiopaque material.

11. The interstitial marker according to claim 1 wherein the plurality of particles include a material visible under ultrasound imaging.

12. The interstitial marker according to claim 1 wherein the plurality of particles include a material visible under diagnostic x-ray.

13. The interstitial marker according to claim 1 wherein the plurality of particles include a material visible under high energy x-ray.

14. The interstitial marker according to claim 1 wherein the plurality of particles include a biocompatible material.

15. A method for creating an interstitial marker in a mammalian body comprising the steps of:
loading a syringe with a fluid marker material;
inserting a needle of the syringe into the mammalian body so that a tip of the needle is at a desired location for the marker; and
injecting the fluid marker material into the mammalian body through the needle.

16. The method for creating an interstitial marker according to claim 15, wherein the fluid marker material includes:
a plurality of particles visible under at least one imaging modality; and
a fluid in which the plurality of particles are suspended.

17. The method for creating an interstitial marker according to claim 15 further comprising the step of moving the tip of the needle during the injecting step.

18. The method for creating an interstitial marker according to claim 17 wherein the moving step includes the steps of:
moving the tip of the needle at a first rate during a first period of time; and
moving the tip of the needle at a second rate during a second period of time.

19. The method for creating an interstitial marker according to claim 17 wherein the injecting step includes the steps of:
injecting the fluid marker at a first flow rate during a first period of time; and
injecting the fluid marker at a second flow rate during a second period of time.
